(19) United States
(12) Patent Application Publication
(10) Pub. No.: US 2023/0059947 A1
(43) Pub. Date: Feb. 23, 2023

(54) SYSTEMS AND METHODS FOR AWAKENING A USER BASED ON SLEEP CYCLE

(71) Applicant: Optum, Inc., Minnetonka, MN (US)

(72) Inventors: Raghav Bali, Delhi (IN); Ninad D. Sathaye, Bangalore (IN); Swapna Sourav Rout, Bangalore (IN)

(21) Appl. No.: 17/444,783

(22) Filed: Aug. 10, 2021

(51) Int. Cl. A61B 5/00; A61B 5/08; A61B 5/11; A61B 5/024; A61B 5/01; A61B 5/16; A61B 5/021; A61M 21/00

(52) U.S. Cl. CPC A61B 5/4812; A61B 5/7267; A61B 5/746; A61B 5/08; A61B 5/11; A61B 5/024; A61B 5/01; A61B 5/163; A61B 5/021; A61M 21/00; A61M 2021/0083

(57) ABSTRACT

A method for managing sleep of a user comprises obtaining, by a computing system, sleep data and environmental data for the user; determining, by the computing system, a sleep state of the user based on the sleep data; determining, by the computing system, one or more awakening actions based on the sleep state of the user and the environmental data; and causing one or more devices in an environment of the user to perform the one or more awakening actions to awaken the user.

### TECHNICAL FIELD

This disclosure relates to sleep assistance and awakening devices.

### BACKGROUND

How a person is awakened from sleep can have a significant impact on the person's mood and decision-making capabilities. For example, being abruptly awakened from a deep sleep can lead to poor decision-making. However, common ways of awakening people, such as alarm clocks and phone-based alarms, will output the same alarm sounds under all conditions.

### SUMMARY

This disclosure describes techniques that may improve systems for helping users stay asleep and for helping users awaken. As described in this disclosure, a computing system may determine a sleep state of a user. In some examples, the computing system may determine one or more sleep-assistance actions based on the sleep state of the user and environmental data regarding an environment of the user. The computing system may cause one or more output devices in the environment of the user to perform the one or more sleep-assistance actions to help keep the user asleep. In some examples, the computing system determines one or more awakening actions based on the sleep state of the user and the environmental data. The computing system may cause one or more output devices in the environment of the user to perform the one or more awakening actions to awaken the user. Because the awakening actions are determined based on the sleep state of the user, the awakening actions may be tailored to help the user awaken in a better mental state.

In one aspect, this disclosure describes a method for managing sleep of a user, the method comprising: obtaining, by a computing system, sleep data and environmental data for the user; determining, by the computing system, a sleep state of the user based on the sleep data; determining, by the computing system, one or more awakening actions based on the sleep state of the user and the environmental data; and causing, by the computing system, an output device in an environment of the user to perform the one or more awakening actions to awaken the user.

In another example, this disclosure describes a computing system comprising: one or more storage devices configured to store sleep data and environmental data for a user; and processing circuitry configured to: determine a sleep state of the user based on the sleep data; determine one or more awakening actions based on the sleep state of the user and the environmental data; and cause an output device in an environment of the user to perform the one or more awakening actions to awaken the user.

In another example, this disclosure describes a non-transitory computer-readable storage medium having instructions stored thereon that, when executed, cause processing circuitry to: obtain sleep data and environmental data for the user; determine a sleep state of the user based on the sleep data; determine one or more awakening actions based on the sleep state of the user and the environmental data; and cause an output device in an environment of the user to perform the one or more awakening actions to awaken the user.

The details of one or more aspects of the disclosure are set forth in the accompanying drawings and the description below. Other features, objects, and advantages of the techniques described in this disclosure will be apparent from the description, drawings, and claims.

### BRIEF DESCRIPTION OF DRAWINGS

FIG. 1 is a block diagram illustrating an example system in accordance with one or more aspects of this disclosure.

FIG. 2 is a block diagram illustrating an example computing system in accordance with one or more aspects of this disclosure.

FIG. 3 is a conceptual diagram illustrating an example recurrent neural network (RNN) architecture in accordance with one or more aspects of this disclosure.

FIG. 4 is a block diagram illustrating an example sleep assistance unit in accordance with one or more aspects of this disclosure.

FIG. 5 is a conceptual diagram illustrating an example of patterned sleep disturbance prediction, in accordance with one or more techniques of this disclosure.

FIG. 6 is a flowchart illustrating an example operation of a processing system in accordance with one or more aspects of this disclosure.

### DETAILED DESCRIPTION

Timely falling asleep, staying asleep, and timely waking from sleep are problems experienced by many people. Existing systems to help people fall asleep and stay asleep include noise-masking systems that generate sound throughout the user's sleep period or at the beginning of the user's sleep period. The generated sound may mask out environmental sound by generating a more consistent sound level. However, long-term exposure to such sound may have negative consequences. At the same time, if a noise-masking system only generates sound during the first part of the user's sleep period, the noise-masking system may not be able to help the user stay asleep.

Moreover, existing systems for helping people awaken from sleep typically involve the generation of an abrupt noise, such as an alarm sound, at a particular time. These existing systems essentially attempt to awaken the user as quickly as possible. However, being abruptly awoken can result in grogginess, poor decision-making, or make it more likely that the user snoozes the alarm. Other systems for helping to awaken users, such as lights that gradually increase in intensity in advance of a fixed wake-up time, may not be sufficient to reliably awaken the users. Moreover, systems for helping awaken users are not adapted to individual locations.

This disclosure describes techniques that may provide one or more technical improvements to systems that help users fall asleep, stay asleep, and/or awaken from sleep. As described herein, a computing system may obtain sleep data and environmental data for a user. The sleep data may provide information about the sleep of the user, such as respiration data, movement data, cardiac data, and so on. The environmental data may include data regarding the environment of the user, such as the temperature, noise level, humidity level, illumination level, and so on. The computing system may determine a sleep state of the user based on the sleep data. Example sleep states may include very light sleep without rapid eye movement (REM) (i.e., sleep stage 1), light sleep without REM (i.e., sleep stage 2), deep sleep without REM (i.e., sleep stage 3), light sleep with REM (i.e., sleep stage 4), and so on.

The computing system may determine one or more actions based on the sleep state of the user and the environmental data. In some examples, the computing system may determine one or more actions to help keep the user asleep. For instance, the actions to help keep the user asleep may include increasing or decreasing masking noises, changing the temperature, and so on. Because the actions to keep the user asleep are based on the user's sleep state, the computing system may be able to use masking noise only when the user is in a sleep state (e.g., light sleep or very light sleep) in which the masking noise may be needed to mask environmental noise or distract the user, thereby reducing the amount of masking noise to which the user is exposed.

In some examples, the computing system may determine one or more actions to awaken the user. Example actions to awaken the user may include gradually increasing the light level, temperature, or sound level in the environment of the user based on the user's sleep state. The computing system may then cause one or more output devices in the environment of the user to perform the actions. Because the computing system may determine the actions to awaken the user based on the user's sleep state, the computing system may be able to transition the user into a sleep state from which the user can awaken more easily, before ultimately waking the user.

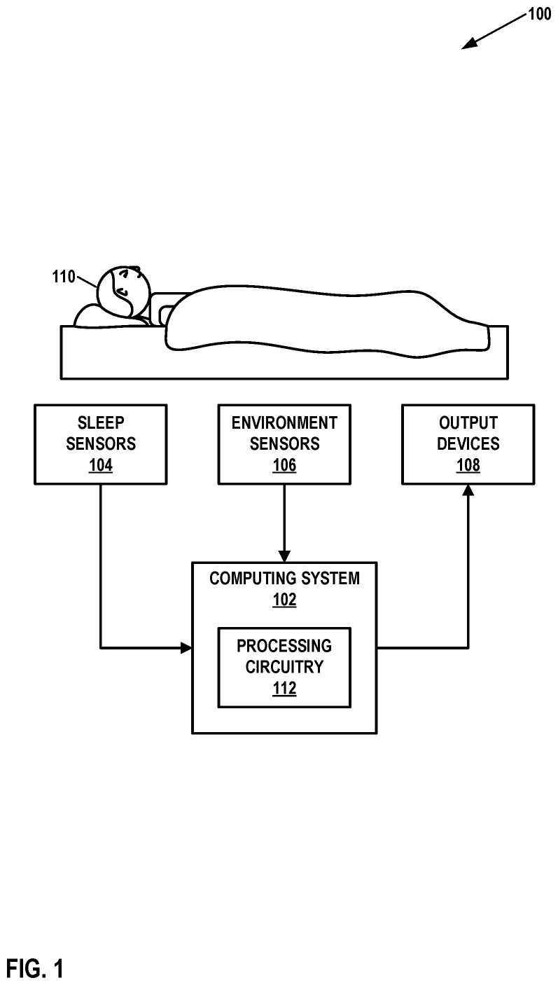

FIG. 1 is a block diagram illustrating an example system 100 in accordance with one or more aspects of this disclosure. In the example of FIG. 1, system 100 includes a computing system 102, one or more sleep sensors 104, one or more environmental sensors 106, and one or more output devices 108. Sleep sensors 104 may generate sleep data regarding a user 110. Environmental sensors 106 may generate environmental data regarding an environment of user 110. Output devices 108 may generate output that may affect the sleep of user 110. User 110 does not form part of system 100.

Computing system 102 may include processing circuitry 112. Processing circuitry 112 may include one or more microprocessors, application-specific integrated circuits (ASICs), field-programmable gate arrays (FPGAs), or other types of processing circuits. Processing circuitry 112 may be distributed among one or more devices of computing system 102. The devices of computing system 102 may include laptop computers, desktop computers, mobile devices (e.g., mobile phones or tablets), server computers, or other types of devices. One or more of the devices of computing system 102 may be local or remote from user 110. In some examples, one or more devices of computing system 102 may include one or more of sleep sensors 104, environmental sensors 106, or output devices 108. This disclosure may describe processing tasks performed by processing circuitry 112 as being performed by computing system 102.

As noted above, sleep sensors 104 may generate sleep data that provides information about the sleep of user 110. The sleep data generated by sleep sensors may be time series data. Sleep sensors 104 may include one or more sensors that generate respiration data that describes respiration of user 110. Example sensors that may generate the respiration data may include inertial measurement units (IMUs), pressure sensors (e.g., which may be integrated into a mattress), photoplethysmography (PPG) sensors, microphones, and so on. In some examples, sleep sensors 104 may include sensors that generate movement data that describe movement of user 110. Example sensors that may generate movement data may include IMUs, pressure sensors (e.g., which may be integrated into a mattress), optical or infrared sensors, and so on. In some examples, sleep sensors 104 may include cardiac data that describe cardiovascular activity of user 110. Example sensors that generate cardiac data include IMUs, PPG sensors, and so on. In some examples, sleep sensors 104 may include sensors that generate body temperature data that describes a body temperature of user 110. Example sensors that generate body temperature data include thermometers, infrared sensors, and so on. In some examples, sleep sensors 104 may include sensors that generate blood pressure data that describes a blood pressure of user 110. Example sensors that generate blood pressure data include PPG sensors, oscillometric sensors, ballistocardiogram sensors, and so on. In some examples, sleep sensors 104 may include sensors that generate ocular movement data that describe ocular movements of the user. Example sensors that generate ocular movement data include IMUs, electromyography (EMG) sensors, optical or infrared sensors, and so on.

In some examples, sleep sensors 104 include electroencephalogram (EEG) sensors. In some examples where sleep sensors 104 include EEG sensors, computing system 102 may use some or all available EEG waveforms. For instance, computing system 102 may use 2-5 EEG waveforms from among 10-12 available EEG waveforms. In some examples, sleep sensors 104 include blood oxygenation sensors. In other words, sleep sensors 105 may include one or more pulse oximeters to measure a peripheral oxygen saturation (SpO₂) of user 110.

Sleep sensors 104 may be included in various types of devices or may be standalone devices. For instance, one or more of sleep sensors 104 may be included in a wearable device (e.g., an ear-wearable device, an earbud, a smart watch, etc.), a smart speaker device, an Internet of Things (IoT) device, and so on.

Environmental sensors 106 may generate data regarding an environment of user 110. Environmental sensors 106 may include ambient light level sensors, temperature sensors, microphones to measure noise, humidity sensors, oxygen level sensors, and so on. In some examples, the same device may include two or more sleep sensors 104, two or more environmental sensors 106, or combinations of one or more sleep sensors 104 and environmental sensors 106. For example, a wearable device (e.g., smartwatch, patch, earphones, etc.) of user 110 may include one or more sleep sensors 104 and one or more of environmental sensors 106.

Computing system 102 may determine a sleep state of user 110 based on the sleep data generated by sleep sensors 104. Additionally, computing system 102 may determine one or more actions based on the sleep state of user 110 and the environmental data generated by environmental sensors 106. In some examples, computing system 102 determines one or more actions to help user 110 fall asleep or to keep user 110 asleep. In some examples, computing system 102 determines one or more actions to awaken user 110.

Computing system 102 may cause one or more of output devices 108 to perform the one or more actions. For instance, computing system 102 may cause one or more of output devices 108 to perform one or more actions to help user 110 stay asleep or to awaken user 110. Examples of output devices 108 may include audio devices (e.g., speakers, headphones, earphones, etc.), temperature-control devices (e.g., thermostats, temperature-controlled clothing, temperature-controlled bedding, temperature-controlled mattresses, etc.), haptic devices, lighting devices, and so on. In some examples, output devices 108 may include one or more of sleep sensors 104 and/or one or more of environmental sensors 106. In some examples, one or more devices of computing system 102 may include one or more of output devices 108, sleep sensors 104, and/or environmental sensors 106. In some examples, computing system 102 may communicate with one or more of sleep sensors 104, environmental sensors 106, and output devices 108 via wire-based and/or wireless communication links.

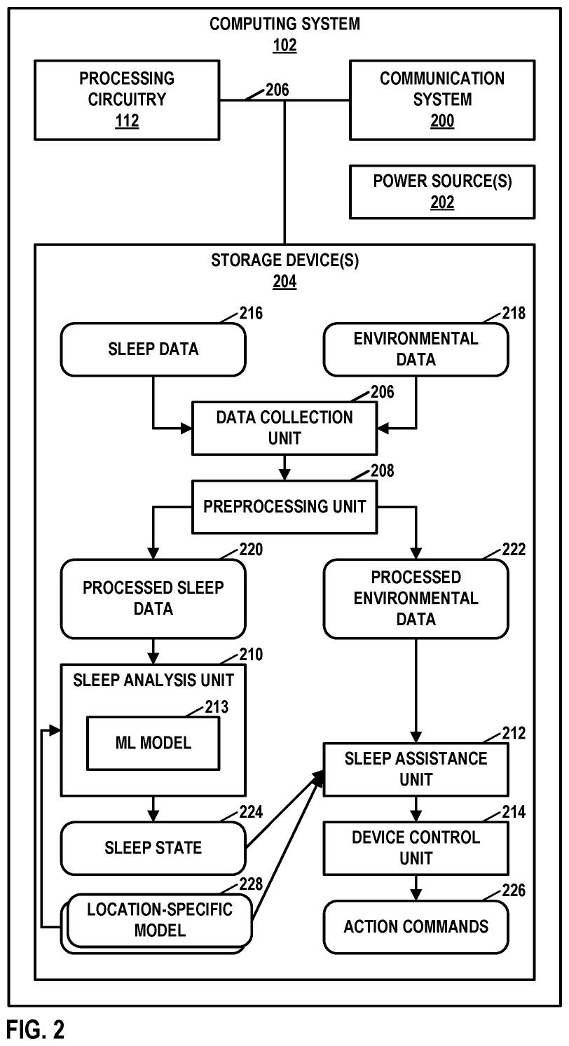

FIG. 2 is a block diagram illustrating example components of computing system 102 in accordance with one or more aspects of this disclosure. In the example of FIG. 2, computing system 102 includes processing circuitry 112, a communication system 200, one or more power sources 202, and one or more storage devices 204. Communication channel(s) 206 may interconnect components of computing system 102 for inter-component communications (physically, communicatively, and/or operatively). In some examples, communication channel(s) 206 may include a system bus, a network connection, an inter-process communication data structure, or any other method for communicating data. Power source(s) 202 may provide electrical energy to processing circuitry 112, communication system 200, and storage device(s) 204. Storage device(s) 204 may store information required for use during operation of computing system 102.

Processing circuitry 112 comprises circuitry configured to perform processing functions. For instance, processing circuitry 112 may include one or more microprocessors, application-specific integrated circuits (ASICs), field-programmable gate arrays (FPGAs), or other types of processing circuits. Processing circuitry 112 may include programmable and/or fixed-function circuitry. In some examples, processing circuitry 112 may read and may execute instructions stored by storage device(s) 204.

Communication system 200 may enable computing system 102 to send data to and receive data from one or more other devices, such as sleep sensors 104, environmental sensors 106, output devices 108, and so on. Communication system 200 may include radio frequency transceivers, or other types of devices that are able to send and receive information. In some examples, communication system 200 may include one or more network interface cards or other devices for cable-based communication.

Storage device(s) 204 may store data. Storage device(s) 204 may include volatile memory and may therefore not retain stored contents if powered off. Examples of volatile memories may include random access memories (RAM), dynamic random access memories (DRAM), static random access memories (SRAM), and other forms of volatile memories known in the art. Storage device(s) 204 may include non-volatile memory for long-term storage of information and may retain information after power on/off cycles. Examples of non-volatile memory may include flash memories or forms of electrically programmable memories (EPROM) or electrically erasable and programmable (EEPROM) memories.

In the example of FIG. 2, storage device(s) 204 store instructions associated with a data collection unit 206, a preprocessing unit 208, a sleep analysis unit 210, a sleep assistance unit 212, and a device control unit 214. Processing circuitry 112 may execute the instructions associated with data collection unit 206, preprocessing unit 208, sleep analysis unit 210, sleep assistance unit 212, and device control unit 214. For ease of explanation, this disclosure describes actions performed by processing circuitry 112 when executing instructions associated with data collection unit 206, preprocessing unit 208, sleep analysis unit 210, sleep assistance unit 212, and device control unit 214 as being performed by data collection unit 206, preprocessing unit 208, sleep analysis unit 210, sleep assistance unit 212, and device control unit 214.

Data collection unit 206 may obtain sleep data 216 generated by sleep sensors 104 and environmental data 218 generated by environmental sensors 106 (e.g., via communication system 200). In some examples, data collection unit 206 may obtain sleep data 216 and/or environmental data 218 in real time as sleep data 216 and environmental data 218 are generated by sleep sensors 104 and environmental sensors 106. Storage device(s) 204 may store sleep data 216 and environmental data 218. In some examples, storage device(s) 204 may store historical values of sleep data 216 and environmental data 218. For instance, in one example, storage device(s) 204 may store, for each time window, a blood pressure value, a heart rate value, a body temperature value, an ambient noise value, and an ambient light value.

Preprocessing unit 208 may process sleep data 216 and environmental data 218 to transform sleep data 216 and environmental data 218 into processed sleep data 220 and processed environmental data 222. For example, preprocessing unit 208 may process sleep data 216 and environmental data 218 to scale features, detect outliers (e.g., outliers caused by sensor miscalibration, outliers caused by data corruption, etc.), impute missing values (e.g., via interpolation), and so on. Scaling of features may be important for some deep learning models that are highly sensitive to the scale of features. In some examples, preprocessing unit 208 may impute missing values using forward-fill or back-fill or statistical measures such as mean or median. In some examples, preprocessing unit 208 may use different processes for imputing missing values for different types of sleep data and or environmental data.

In some examples, preprocessing unit 208 may perform feature engineering to generate derivative features, such as lag and lead indicators. For instance, in one example, preprocessing unit 208 may be a function (e.g., using regression, a linear function, etc.) describing time series data of sleep data 216 and/or environmental data 218. In this example, preprocessing unit 208 may use the function to determine lag indicators (i.e., data points following data points of the time series data) or lead indicators (i.e., data points preceding data points of the time series data).

In some examples, preprocessing unit 208 may normalize sleep data 216 and/or environmental data 218, e.g., by applying a Short Time Fourier Transform (STFT) to sleep data 216 and/or environmental data 218. The STFT may transform features of sleep data 216 and/or environmental data 218 into a more useful hyperspace or feature space. The STFT may preserve time-ordering of frequencies observed in signals in sleep data 216 and/or environmental data 218. In some examples, preprocessing unit 208 may normalize sleep data 216 and/or environmental data 218 may applying a wavelet transformation to one or more features of sleep data 216 and/or environmental data 218. Application of the wavelet transformation may be suitable for features where time-frequency locations is important.

In the example of FIG. 2, sleep analysis unit 210 may use processed sleep data 220 (and in some examples, processed environmental data 222) to determine a sleep state of user 110 for a specific time window (e.g., a current or historic time window). In some examples, sleep analysis unit 210 may determine whether user 110 is in a REM state for a specific time window (e.g., a current or historic time window).

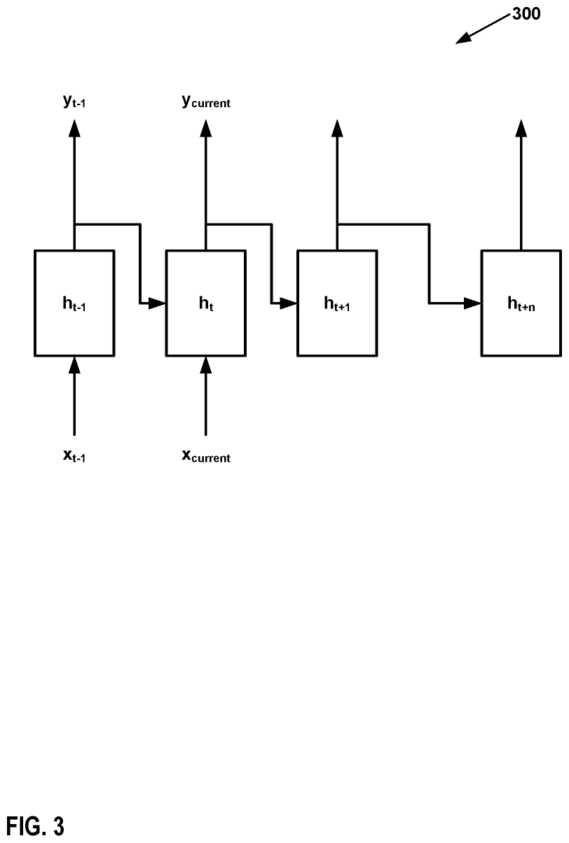

In some examples, sleep analysis unit 210 includes a machine-learned (ML) model 213. In some examples where sleep analysis unit 210 includes ML model 213, ML model 213 includes a neural network, such as a recurrent neural network (RNN). FIG. 3 is a conceptual diagram illustrating an example RNN architecture 300 in accordance with one or more aspects of this disclosure. In the example of FIG. 3, blocks 302A through 302N correspond to a same neural network at different time instances (labeled t-1 through t+n). For each time instance after a first time instance, the neural network receives input data (x) for the time instance and output (y) of the neural network for the previous time instance. For the first time instance, a set of default values may be used as input in place of the output of the neural network for the previous time instance.

With respect to sleep analysis unit 210, the neural network may include output neurons corresponding to potential sleep states (e.g., combinations of sleep stages and REM state). Thus, in some examples, the output of the neural network may be a set of confidence values that indicate levels of confidence that user 110 is in the corresponding sleep state for a specific time window. In some examples, the neural network has output neurons for different sleep states. In some examples, the neural network has output neurons for different sleep stages and different REM states. Sleep analysis unit 210 may determine that a sleep state 224 (e.g., sleep stage and/or REM state) of user 110 as the sleep state corresponding to the output neuron that produced the greatest output value. Storage device(s) 204 may store data indicating the sleep state 224 of user 110 for the specific time window.

Sleep states of user 110 may be influenced by physiological and lifestyle parameters. Accordingly, in some examples, sleep analysis unit 210 may use information in addition to processed sleep data 220 to determine sleep state 224. For instance, in some examples, sleep analysis unit 210 may user information regarding food intake and/or physical activity of user 110, in addition to processed sleep data 220 to determine sleep state 224. For instance, physical exercise plays an important role in deciding the quality of sleep. Physical activity may also have a direct impact on the duration of different sleep stages. Physical activity may be directly measured by wearable devices, such as smart watches, mobile phones, ear-worn devices, or other devices. Sleep analysis unit 210 may also obtain information regarding timing of the physical exercise (e.g., morning, night, or day), total time of exercise, strenuous level of the exercise, and/or other information regarding exercise of user 110. Information regarding exercise of user 110 may be determined from IMU sensors of wearable devices. Sleep analysis unit 210 may obtain various types of information regarding food intake of user 110. For instance, the information regarding food intake of user 110 may include timing and quantity of the food (e.g., light/heavy). Computing system 102 may obtain information regarding food intake of user 110 (e.g., from user 110 via a user interface) prior to the start of a start of a sleep period of user 110.

In addition to exercise and/or food intake information for a current day, sleep analysis unit 210 may use historical data regarding exercise and/or food intake to determine sleep state 224. For example, sleep analysis unit 210 may use one or more statistics regarding exercise and/or food intake of user 110 as input to ML model 213 to determine sleep state 224. For instance, in an example where ML model 213 includes a neural network, the neural network may include different input neurons for different statistics regarding exercise and/or food intake of user 110.

Furthermore, in the example of FIG. 2, sleep assistance unit 212 may determine one or more actions based on the sleep state 224 of user 110 and processed environmental data 222. For example, sleep assistance unit 212 may determine one or more actions to help user 110 fall asleep or stay asleep. In some examples, sleep assistance unit 212 may determine one or more actions to awaken user 110. In the example of FIG. 2, storage device(s) 204 may also store one or more location-specific models 228. Sleep assistance unit 212 may use location-specific models 228 to predict potential sleep disturbances, determine occurrences of alarm events, determine actions to help user 110 fall asleep, stay asleep, or awaken, and/or other purposes.

Device control unit 214 may cause one or more of output devices 108 to perform the one or more actions. For instance, device control unit 214 may cause one or more output devices 108 to perform actions to help user 110 fall asleep or stay asleep. In some examples, device control unit 214 may cause one or more output devices 108 to perform actions to awaken user 110. To cause the one or more output devices 108 to perform actions, device control unit 214 may send commands or other messages to the one or more output devices 108. In some examples, device control unit 214 may use communication system 200 to send the commands to the one or more output devices 108. In examples where computing system 102 includes an output device, device control unit 214 may cause the output device to perform one or more actions without using communication system 200 or other inter-device communication.

In some examples, computing system 102 may perform a user identification process. For instance, different users may have different sleeping profiles and preferences. Moreover, the sleep data for different users may be different despite the users being in the same sleep state. Accordingly, computing system 102 may receive data indicating an identity of user 110. Based on the identity of user 110, computing system 102 may select models (e.g., a model for determining a sleep state, a model for unprovoked sleep disturbances, a model for selecting actions, and so on) specifically for user 110. The models used for a specific user (e.g., user 110) may be part of a profile for the specific user.

In some examples where computing system 102 performs a user identification process and output devices 108 include a user-specific device (e.g., earbuds), computing system 102 may obtain information regarding the availability of the user-specific device, e.g., from other devices in an environment, such as smart home devices or a mobile phone of user 110. In such examples, a smart home device, such as a smart speaker, may wirelessly scan for availability of the user-specific device. If the smart home device detects the user-specific device, the smart home device may audibly prompt user 110 to confirm their identity. For example, the smart home device may ask “Are you John Smith?” Upon receiving confirmation of the identity of user 110, computing system 102 may start use of an existing profile for user 110. In some examples, the smart home device may use voice recognition technology to confirm the identity of user 110. If user 110 is not associated with an existing profile, computing system 102 may use a default profile that is not customized to any specific user. In some examples where the same device is used by different people, a button on the device or one or more graphical interface controls on a device (e.g., mobile phone) may be used to indicate which user is using the device.

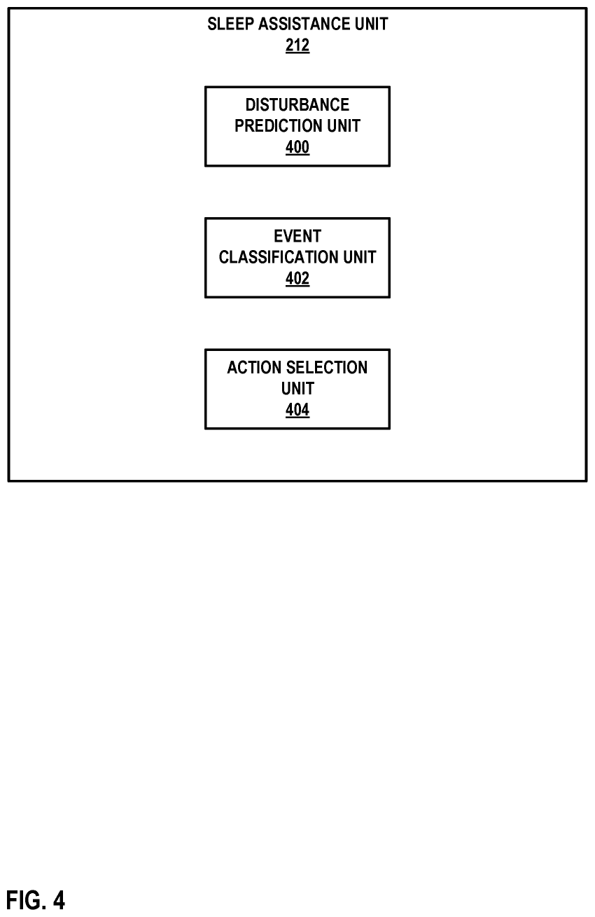

FIG. 4 is a block diagram illustrating an example sleep assistance unit 212 in accordance with one or more aspects of this disclosure. In the example of FIG. 4, sleep assistance unit 212 may include a disturbance prediction unit 400, an event classification unit 402, and an action selection unit 404. Disturbance prediction unit 400 may predict occurrences of events that may disturb the sleep of user 110. Such events may be unprovoked events or patterned events. Unprovoked events may be events that are not provoked by environmental factors. Patterned events may be events that recur according to patterns in an environment of user 110. For instance, as an example of a patterned event, environmental noise levels may increase according to a schedule, e.g., such as when a train passes by each night or when road traffic starts to increase in the morning.

A patterned sleep disturbance can be an external event that regularly occurs around a specified time. For instance, in a given locality, there may be a garbage collection truck that arrives for pickup every night at 4 am. The garbage collection truck may create a disturbance at the same time every night and disturb the sleep of user 110. Patterns of events in the environment of user 110 that disturb the sleep of user 110 may be relatively less frequent in the span of sleep duration as compared to sleep state transitions. While the sleep state transitions may be predicted by sleep analysis unit 210 at a frequency of, e.g., 5-10 milliseconds, patterned sleep disturbances may involve a broader analysis of sleep duration.

Disturbance prediction unit 400 may monitor the sleep state 224 and processed environmental data 222 to detect patterned events. For example, disturbance prediction unit 400 may implement a machine-learning model that takes, as input, processed environmental data 222 and a time indicator. The machine-learning model may output a prediction indicating whether user 110 is experiencing a sleep disruption. Disturbance prediction unit 400 may train the machine-learned model based on a comparison of the prediction with the sleep state 224. For instance, in an example where the machine-learning model is a neural network, disturbance prediction unit 400 may apply an error function that takes the prediction and the sleep state 224 as inputs and produces an error value. Disturbance prediction unit 400 may then use the error value in a backpropagation algorithm to update parameters of the neural network.

In some examples, disturbance prediction unit 400 may identify a patterned sleep disturbance using a Sequence-to-Sequence prediction model. The Sequence-to-Sequence prediction model may take the events of an entire sleep period (e.g., 8-10 hours) as a single snapshot to predict sleep disturbance intervals. As part of predicting sleep disturbance intervals, disturbance prediction unit 400 may divide a sleep period into multiple time windows. The Sequence-to-Sequence prediction model may be or include a recurrent neural network (RNN) that receives an input sequence as input. The input sequence may include multi-variate time series sensor data for a time window during the sleep period. For each respective time period, input sequence provided to the RNN, the RNN produces, based on the input sequence for the respective time period (and, in some examples, one or more of the time periods preceding the respective time period), one or more dependent variables that indicate whether user 110 will be asleep or awake and a sleep state of user 110 during one or more time periods that follow the respective time period (e.g., future time periods). Disturbance prediction unit 400 may include the one or more dependent variables in the input sequence for a next time window. The RNN may be trained using data from multiple sleep periods so that the RNN may predict whether user 110 is likely to be awake or sleep and/or the sleep state of user 110 during different time windows during a sleep period.

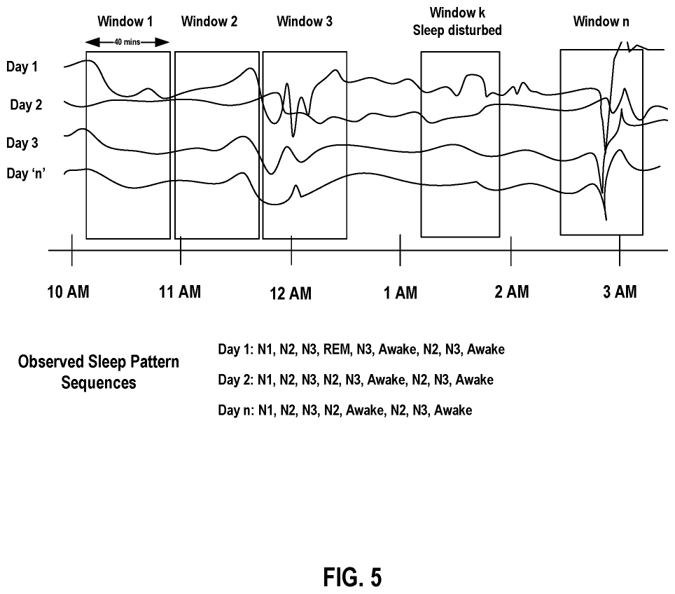

FIG. 5 is a conceptual illustrating an example of patterned sleep disturbance prediction, in accordance with one or more techniques of this disclosure. In the example of FIG. 5, a sleep period is divided into 40-minute time windows, however time windows of other durations may be used. The lines in FIG. 5 correspond values generated by a sensor (e.g., a temperature sensor) during different days. The lines in FIG. 5 are vertically separated to reduce overlap of the lines. In some implementations, disturbance prediction unit 400 may use data from multiple sensors to predict sleep disturbances. Disturbance prediction unit 400 may generate a prediction whether a sleep disturbance will occur during a specific time window one or more time windows (e.g., 3-4 time windows) in advance of the specific time window.

For each of the time windows, disturbance prediction unit 400 may assign a label to the time window. The label may indicate a sleep state or whether user 110 is awake. Disturbance prediction unit 400 may assign a label to a time window based on the output of the Sequence-to-Sequence model for the time window. For example, the output of the Sequence-to-Sequence model for a time window may include numerical values associated with different sleep states (including an awake state). In this example, disturbance prediction unit 400 may use the numerical values generated by the Sequence-to-Sequence model for the time window to assign a label to the time window. For instance, disturbance prediction unit 400 may identify a highest one of the numerical values and use a table that maps output values of the Sequence-to-Sequence model to labels to determine a label mapped to the highest one of the numeral values. The table mapping output values to labels may be based on empirical data generated by observations or a laboratory or other setting. In some examples, unsupervised learning may be used to generate the table.

Disturbance prediction unit 400 may concatenate the labels to form a label sequence. Disturbance prediction unit 400 may use label sequences for multiple sleep periods to predict whether a time window that is a given number of time windows (e.g., 1 time window, 2 time windows, 3 time windows, etc.) after a current time window would be labeled as “awake.” For instance, disturbance prediction unit 400 may determine based on the distribution of labels assigned to a time window which label is most probable for the time window. For example, the time periods may include a first time period corresponding to 1:30 am to 2:10 am, a second time period corresponding to 2:10 am to 2:50 am, and a third time period corresponding to 2:50 am to 3:30 am. In this example, disturbance prediction unit 400 may determine, based on the labels assigned to these time windows over the course of several sleep periods, that the most common label for the first time period is asleep, that the most common label for the second time period is awake, and that the most common label for the third time period is asleep. Thus, disturbance prediction unit 400 may for a label sequence of asleep-awake-asleep for these three time periods. Therefore, disturbance prediction unit 400 may determine that user 110 is likely to be awake most nights between 2:10 am and 2:50 am.

Thus, based on the output of the sequence-to-sequence model, disturbance prediction unit 400 may determine, on a recurrent basis (e.g., every 40 minutes), a label to assign to a current time window. Based on the labels assigned to the current time window and previous time windows, disturbance prediction unit 400 may determine whether one or more future time windows will be labeled as “awake.” If disturbance prediction unit 400 determines that a future time window is likely to be assigned the label of “awake,” disturbance prediction unit 400 may instruct action selection unit 404 to select one or more sleep-assistance actions.

To help ensure undisturbed sleep, disturbance prediction unit 400 may also predict unprovoked sleep disturbances. For example, sleep assistance unit 212 may cause device control unit 214 to gradually lower output volume of sleep assistance sounds as user 110 progresses through a series of sleep states. In this example, if disturbance prediction unit 400 determines that an unprovoked sleep disturbance may occur in an upcoming time window (e.g., the next time window), sleep assistance unit 212 may cause device control unit 214 to gradually increase the output volume of the sleep assistance sounds to help keep user 110 asleep during the predicted unprovoked sleep disturbance.

In some examples, to predict an unprovoked sleep disturbance, disturbance prediction unit 400 may obtain processed sleep data 220 and sleep state 224. In this example, disturbance prediction unit 400 may apply a machine-learned model (e.g., the Sequence-to-Sequence model) that generates a prediction regarding whether user 110 will experience a sleep disturbance in a future time window (e.g., in a time window 5, 10, 15, etc. minutes from the current time). Furthermore, in this example, input to the machine-learned model may include processed sleep data 220. In some examples, input to the machine-learned model may also include other data, such as data indicating a current time. Disturbance prediction unit 400 may train the machine-learned model based on the determined sleep state 224. In this way, disturbance prediction unit 400 may predict unprovoked sleep disturbances based on historical sleep data. For instance, in an example where the machine-learned model is a neural network, disturbance prediction unit 400 may apply an error function that takes the prediction and the sleep state 224 as inputs and produces an error value. Disturbance prediction unit 400 may then use the error value in a backpropagation algorithm to update parameters of the neural network. In this way, disturbance prediction unit 400 may be able to predict, based on processed sleep data 220 (e.g., vital signs of user 110, etc.), that user 110 will experience a sleep disturbance. Predicting and responding to such unprovoked sleep disturbances may be helpful for users who spontaneously awaken during their planned sleep periods. For instance, user 110 may spontaneously awaken around 3:00 am or after having certain types of sleep conditions (e.g., intense dreams) and may have difficulty getting back to sleep.

In some examples, disturbance prediction unit 400 may present information regarding patterned sleep disturbances and/or unprovoked sleep disturbances to user 110 for validation. For instance, disturbance prediction unit 400 may prompt user 110 to validate whether user 110 experienced an unprovoked sleep disturbance during the sleep period or during a specific time window during the sleep period. Similarly, in some examples, disturbance prediction unit 400 may prompt user 110 to validate whether user 110 has experienced a patterned sleep disturbance. For instance, disturbance prediction unit 400 may prompt user 110 to indicate whether the sleep of user 110 has been disturbed by ambient noise around 2:00 am on weekday nights. Additionally, disturbance prediction unit 400 may obtain data from user 110 indicating that user 110 experienced a sleep disruption, and in some examples, a time window during which user 110 experienced the sleep disruption. Disturbance prediction unit 400 may include data obtained from user 110 regarding whether user 110 experienced a sleep disturbance in a user profile for user 110.

Disturbance prediction unit 400 may train machine-learned models for predicting sleep disturbances based on the responses of user 110 to the requests from disturbance prediction unit 400 for validation and/or indication from user 110 regarding sleep disruptions. For example, if a machine-learned model for predicting unprovoked sleep disturbances did not predict an unprovoked sleep disturbance during a time window, but user 110 experienced a sleep disturbance during the time window, disturbance prediction unit 400 may update parameters of the machine-learned model to increase a likelihood that the machine-learned model will predict an unprovoked sleep disturbance given sleep state 224, sleep data, and/or other information applicable to the time window. In some examples, if a machine-learned model for predicting a patterned sleep disturbance predicted a patterned sleep disturbance for a time window and user 110 validated the occurrence of the patterned sleep disturbance during the time window, disturbance prediction unit 400 may further modify parameters of the machine-learned model to increase a confidence of a patterned sleep disturbance given environmental data and sleep state for the time window.

Event classification unit 402 may determine whether to perform an intervention. Given the data regarding a current sleep stage of user 110 and a predicted or actual event, event classification unit 402 may predict whether user 110 will experience a sleep disturbance. Event classification unit 402 may then determine, based on this prediction, whether to perform an intervention (i.e., whether to cause one or more of output devices 108 to perform one or more actions).

In some examples, event classification unit 402 may determine whether an alarm event is occurring. An alarm event may be an event that requires user 110 to awaken. Examples of alarm events may include health emergencies, alarm conditions, detection of a baby crying, detection that a person (e.g., a dementia patient) has left a designated area or otherwise needs assistance, and so on. When an alarm event occurs, event classification unit 402 may cause output devices 108 to stop performing sleep-assistance actions and/or may cause output devices 108 to perform actions to awaken user 110. Event classification unit 402 may determine on a periodic basis whether an alarm event is occurring. For instance, event classification unit 402 may determine every 30-50 milliseconds (ms) whether an alarm event is occurring.

Event classification unit 402 may determine whether an alarm event is occurring in a variety of ways. For example, event classification unit 402 may interact with one or more external systems (e.g., via APIs or other interfaces) to obtain information that event classification unit 402 uses to determine whether an alarm event is occurring. For example, event classification unit 402 may obtain cardiac rhythm data regarding a heart rhythm of a person (e.g., user 110 or another person) from one or more sensors (e.g., sleep sensors 104 or other sensors). In this example, event classification unit 402 may determine, based on the cardiac rhythm data, that an alarm event is occurrent when the person is experiencing a dangerous cardiac arrhythmia. In another example, event classification unit 402 may obtain alarm data from an alarm system (e.g., a security alarm system, an equipment alarm system, a smoke or carbon monoxide alarm system, etc.). In this example, event classification unit 402 may determine that an alarm event is occurring if the alarm data indicates that an alarm event is occurring. In another example, event classification unit 402 may determine that an alarm event is occurring when a baby monitor detects that a baby is crying or making other sounds. In another example, event classification unit 402 may determine that an alarm event is occurring if an epilepsy monitoring device detects or predicts an onset of an epileptic seizure. In some examples, event classification unit 402 may use alarm condition preferences established for user 110 to determine whether an alarm event is occurring. The alarm condition preference may indicate preferences of user 110 with respect to event alarm events are determined to occur.

Event classification unit 402 may use different location-specific models to determine whether alarm events are occurring. Thus, event classification unit 402 may determine that an alarm event is occurring for user 110 when user 110 is at a first location but determine that no alarm event is occurring for user 110 when user 110 is at a second location, despite there being the same underlying conditions. For example, a nurse may work at an elderly care home in location A and an elderly care home in location B. In this example, if user 110 is working at the elderly care home in location A, event classification unit 402 may determine that an alarm event is occurring if a patient experiences a medical emergency. However, in this example, if user 110 is working at the elderly care home in location B, event classification unit 402 does not determine that an alarm event is occurring if the patient experiences a medical emergency. Event classification unit 402 may receive indications of user preferences that indicate which conditions are to result in alarm events for one or more locations.

As noted above, event classification unit 402 may cause output devices 108 to stop performing sleep-assistance actions when an alarm event is occurring. In an example where the sleep-assistance actions include sound generation, stopping performance of the sleep-assistance actions may include reducing the volume of sleep-assistance sounds generated by output devices 108 to 0. The following pseudo-code may express this example:

```

[symbol]
: SPov = 0 dB
for parameters list {Ws, As, Ct} from t − 1 epoch:
  if (Ctk == 1) :
    SPov = 0 dB
  Else:
    SP0V = f (SSp, As)
```

In the pseudo code above, SPₒᵥ indicates an output volume of a sound generation device, W_{S} indicates processed sleep data 220, A_{S} indicates processed environmental data 222, Cₜ indicates alarm data for the time window t, SSₚ indicates a sleep state 224. Cₜ is equal to 1 if event classification unit 402 determines that an alarm condition is occurring. Furthermore, in the pseudo code above f (SSₚ, A_{S}) is a function that determines the output volume SPₒᵥ.

In one example, user 110 may have an alarm set for 4:00 am. This alarm may be an alarm event. Thus, at 4:00 am, event classification unit 402 may determine that an alarm event is occurring. Thus, event classification unit 402 may cause output devices 108 to cease sleep-assistance activities at 4:00 am. In another example, a housemate of user 110 may use a heartbeat monitor. In this example, if event classification unit 402 (or another device or system) determines that the housemate is experiencing a cardiac arrythmia, event classification unit 402 may cause output devices 108 to cease sleep-assistance activities and may cause output devices 108 to perform one or more actions to awaken user 110.

Action selection unit 404 may select one or more actions for output devices 108 to perform. Action selection unit 404 may select the one or more actions for output devices 108 to perform in response to one or more events. For example, action selection unit 404 may select one or more actions in response to event classification unit 402 determining that an alarm event is occurring. In other words, action selection unit 404 may select one or more actions in response to event classification unit 402 determining that user 110 is required to wake up in a current time window or upcoming time window. In some examples, action selection unit 404 may select one or more actions in response to an indication of user input from user 110 to do so. In some examples, action selection unit 404 may select one or more actions in response to disturbance prediction unit 400 determining that user 110 is experiencing or is likely to experience an unprovoked sleep disturbance in a future time window. In some examples, action selection unit 404 may select one or more actions in response to disturbance prediction unit 400 determining that user 110 is experiencing or is likely to experience a patterned sleep disturbance in a future time window.

Action selection unit 404 may determine, for one or more output devices 108, an output and duration of a sleep-assist action that is linked to a sleep state and environmental conditions. For example, if user 110 is in sleep stage 3 with no REM, user 110 is in deep sleep and brain activity is at a minimum. Accordingly, in this example, action selection unit 404 may reduce the output volume of sleep-assistance sounds to a minimum or perform selective noise cancelation because user 110 is less likely to awaken due to environmental sounds. Reducing the output volume of sleep-assistance sounds and/or more selectively performing noise cancelation may also help to conserve electrical energy, which may be especially significant for battery-powered devices. Reducing the output volume of sleep-assistance sounds and/or more selectively performing noise cancelation may also reduce a noise exposure level of user 110. In another example, if sleep state 224 of user 110 is a REM state, there is a sharp increase in brain activity and user 110 is more prone to being awakened by external disturbances. Accordingly, in this example, action selection unit 404 may increase the output volume and/or increase noise cancelation to help ensure undisturbed sleep.

In some examples, determining an action to perform may comprise determining an output volume of sleep-assistance sounds. In some such examples, action selection unit 404 may use an equation, such as equation 1, below, to determine the output volume.

$${SP}_{ov} = {β}_{0} + {λ}_{SS} {SS}_{p} + {\sum }_{j = 1}^{m} {λ}_{{a}_{j}} {A}_{{s}_{j}} + ε \qquad ( 1 )$$

In equation 1, SPₒᵥ indicates an output volume of a speaker, β₀ indicates a basic volume setting of the speaker, SSₚ indicates a sleep state of user 110 as predicted by sleep analysis unit 210, m indicates a number of environmental variables, A_{S}_{ⱼ} indicates a value of an environment variable (e.g., light intensity in the environment of user 110, ambient noise in the environment of user 110, whether a current time is within a periodic event window (e.g., whether an event is likely to occur in an environment of user 110 at the current time), and so on). In equation 1, the λ values are weights. The λ values may be different for individual users and for different environments or locations. In equation 1, the value ε is an irreducible error. Action selection unit 404 may calculate the output volume on a recurrent basis. For example, action selection unit 404 may calculate the output volume every 5-10 milliseconds using a rolling time window.

In some examples where a first environment variable is an ambient light level, a second environment variable is an ambient noise level, and a third environment variable indicates whether the current time is within a periodic event window, example values of the λ values may be λ_{SS}=0.8, λₐ_{₁}=0.25, λₐ_{₂}=0.75, and λₐ_{₃}=0.25. Thus, in this example, if β₀=2, SSₚ=0.8, A_{S}_{₁}=30 lumens, A_{S}_{₂}=30 dB, A_{S}_{₁}=1, and ε=0.1, the output volume SPₒᵥ may be equal to 33.1 dB.

The λ values may initially be set to pretrained values that are determined during a model training stage. Action selection unit 404 may continue to optimize the λ values over time. Moreover, action selection unit 404 may optimize the λ values for specific users, such as user 110, based on data that are collected over time. The collected data may be user-specific. Because the λ values may be optimized for a specific user, the volume level may be customized to the specific user. In some examples, action selection unit 404 applies a machine learning process to learn the one or more weights for the sleep state and the one or more weights for the environmental data. For example, user 110 or other people may manually set the λ values to limit the impacts individual sensors or groups of sensors. For instance, in this example, user 110 or other person may choose among low, medium, or high sensitivity settings for sensors or groups of sensors, where the low, medium, and high sensitivity settings correspond to different λ values. In some examples, a machine learning model may work in tandem with feedback from user 110. In such examples, the machine learning model may take different λ values along with SPₒᵥ as input and may use user feedback indicating whether user 110 is or is not comfortable to achieve a target variable. For example, action selection unit 404 may use a genetic algorithm or simulated annealing process to determine the λ values.

In some examples, action selection unit 404 may use different λ values for different locations. For example, action selection unit 404 may use a first set of λ values when user 110 is sleeping at a first location and a second set of λ values when user 110 is sleeping at a second location. In some examples, action selection unit 404 may perform a machine learning process, such as that described above, for each location at which user 110 sleeps.

In some examples, action selection unit 404 may learn a λ values (e.g., weights for the sleep state and the weights for the environmental data) using data regarding people other than the user who sleep at the location. For instance, action selection unit 404 may perform a machine learning process similar to that described above but using anonymized data from multiple users who have slept at the location. Thus, action selection unit 404 may use the learned λ values when user 110 first sleeps that the location. Action selection unit 404 may subsequently continue to learn the λ values based on sleep states of user 110. In this way, action selection unit 404 may use the λ values based on other users as a starting point for λ values used when user 110 sleeps at the location.

As an example of adjusting the λ values, user 110 may sleep in an environment where an average ambient sound is relatively high, even during the sleep period of user 110 (e.g., at night). For instance, the house of user 110 may be close to a busy commercial street in a city. In this example, the A value for ambient noise (e.g., λₐ_{₂}) may have a greater impact on the output value SPₒᵥ. Accordingly, the A value for ambient noise may be greater for user 110 than for a user whose house is in a quieter location. In another example, one or more of the λ values associated with wearable sensors may be different for users who use different types of wearable devices. For instance, because of different sensor calibrations, specific λ values may be different for users who use wearable devices from a first brand as compared to users who use wearable devices from a second brand. Furthermore, different users may have different A value because of differences in physical and mental health.

While sleep-assistance sounds may help users fall asleep or stay asleep, sudden changes in the volume of sleep-assistance sounds may have a negative impact on the ability of user 110 to fall asleep or stay asleep. Hence, in accordance with a technique of this disclosure, action selection unit 404 may dynamically adjust the output volume based on the sleep state 224 of user 110 and environmental data 218. In contrast, traditional crossfading or volume adjustment techniques may gradually increase or decrease output volume over a predefined amount of time to a predefined volume level, without consideration of the sleep state or environment of a user.

In some examples, action selection unit 404 may start the output volume at predefined levels for specific time windows and may gradually learn to adjust the output volume based on the inputs to sleep assistance unit 212 (e.g., sleep state 224, processed environmental data 222, etc.). As user 110 progresses through deeper sleep states (e.g., sleep stage 2, 3, or 4), action selection unit 404 may gradually decrease the output volume to near zero. However, in the case of a predicted sleep disturbance, action selection unit 404 may gradually increase the output volume to levels that are comforting to user 110.

Although equation 1 is described primarily with respect to output volume of a sound generation device, action selection unit 404 may use similar equations to determine levels of output parameters of other output devices. Such equations may have different β₀, ε, and λ values than an equation used to determine output volume. For example, action selection unit 404 may use an equation similar to equation 1 to determine a temperature level of a room, temperature-controlled blanket, temperature-controlled mattress, or other temperature-controlled device. In another example, action selection unit 404 may use an equation similar to equation 1 to determine an illumination level. Moreover, in some examples, in addition to or as an alternative to adjusting the output volume, action selection unit 404 may determine a frequency or pitch of the sound generated by one or more sound-generation devices.

In some examples, action selection unit 404 may select sleep-assistance actions and/or awakening actions based on user preferences of user 110. For instance, computing system 102 may receive indications of user input expressing user preferences. Example types of user preferences may include types of sleep-assisting sounds or awakening sounds. For instance, the preferred sleep-assisting sounds of user 110 may be the sound of waves on a beach. The preferred awakening sounds of user 110 may be the sound of chirping birds.

In some examples, the preferences of user 110 may indicate events for which user 110 does and does not want to be awoken. For instance, a partner of user 110 may arrive home from work at a specific time (e.g., 3 am) during the sleep period of user 110. Arrival of the person may be detected by environmental sensors 106. The preferences of user 110 may indicate that user 110 does not wish to be awoken by the event of the partner of user 110 arriving home from work. Accordingly, action selection unit 404 may select sleep-assistance actions to help keep user 110 asleep when the partner of user 110 arrives home from work. Conversely, in some examples, the preferences of user 110 may indicate that user 110 does wish to be awoken when the partner of user 110 arrives home from work.

In some examples, action selection unit 404 may include a machine-learned model that may be specific to user 110 and that is trained to predict an action, such as a sleep-assisting sound, an awakening sound, a change of temperature, etc.) or other action based on sleep state 224, processed environmental data 222, and/or other data. For example, action selection unit 404 may generate statistics about the effectiveness of different actions in a plurality of different actions for keeping user 110 or waking user 110 for different sleep states and environmental conditions. In this example, action selection unit 404 may select, based on the statistics for the sleep states and environmental conditions, an action that is most likely to keep user 110 asleep or to awaken user 110. In some examples, the machine-learned model may include a neural network that takes sleep state 224 and processed environmental data 222 as input. In this example, the neural network may generate output values for different available actions in a plurality of actions. Action selection unit 404 may train the neural network based on the statistical data. For instance, action selection unit 404 may calculate error values based on a difference between output values of the neural network and a most-probable action. Action selection unit 404 may use the error values in a backpropagation algorithm to update parameters of the neural network.

Action selection unit 404 may use different machine-learned models for different types of output devices 108. For instance, action selection unit 404 may use a first machine-learned model to predict actions for a first output device and a second machine learned model to predict actions for a second output device. In other examples, action selection unit 404 may use a single machine-learned model to predict actions for multiple output devices.

As noted elsewhere in this disclosure, sleep assistance unit 212 may use location-specific models 228 as part of a process to determine sleep-assistance actions and/or awakening actions. For instance, in some examples, disturbance prediction unit 400 may use location-specific models 228 to determine patterned sleep disturbances associated with specific locations.

In some examples, sleep assistance unit 212 may obtain information indicating a location. For instance, sleep assistance unit 212 may receive an indication of user input from user 110 to indicate a location of user 110. In some examples, sleep assistance unit 212 may receive information indicating a location from one or more devices, such as devices that include one or more of sleep sensors 104, environmental sensors 106, and/or output devices 108.

In some examples, sleep assistance unit 212 may train location-specific models 228 based on sleep data from a plurality of people. For example, a location-specific model may be associated with a particular location, such as a particular dormitory, hotel room, or bedroom. In this example, different people may sleep in the particular location. Hence, sleep assistance unit 212 may obtain sleep states for multiple different people along with corresponding environmental data. The data obtained by sleep assistance unit 212 may be anonymized for privacy. Sleep assistance unit 212 may train the location-specific model associated with the particular location based on the obtained data. Thus, disturbance prediction unit 400 may use the location-specific model associated with the particular location to predict patterned sleep disturbances experienced by users that sleep in the particular location. For instance, if a train passes near the particular location at a specific time of day, the location-specific model associated with the particular location may predict that users are likely to experience sleep disruptions at the specific time of day.

When a user (e.g., user 110) starts occupying the particular location, disturbance prediction unit 400 may initially use the location-specific model associated with the particular location. In some examples, disturbance prediction unit 400 may generate a separate copy of the location-specific model associated with the particular location that is customized for the user. In other words, the location-specific model associated with the particular location can serve as a default location-specific model associated with the particular location, which can further serve as a starting point for generating a location-specific model associated with the particular location that is customized for the user. For instance, if the sleep of the user is disrupted by an environmental event that typically does not disturb other users, disturbance prediction unit 400 may train the location-specific model associated with the particular location that is customized for the user to predict that the sleep of the user will be disrupted by the environmental event. For instance, disturbance prediction unit 400 may implement the Sequence-to-Sequence model that takes sequence of processed sleep data 220 and/or processed environmental data 222 for a current time window (and, in some examples, one or more time windows previous to the current time window) to predict sleep states for one or more future time windows. Disturbance prediction unit 400 may later use sleep state data determined by sleep analysis unit 210 for those future time windows, along with process sleep data 220 and/or processed environmental data 222 for those future time windows, as training data to train the Sequence-to-Sequence model to customize the Sequence-to-Sequence model for the user.

In some examples, one or more of location-specific models 228 may be associated with types of locations, e.g., as opposed to individual locations. For instance, specific location-specific models 228 may be associated with locations in suburban areas and other location-specific models 228 may be associated with locations in urban areas.

Furthermore, there may be different types of sleep sensors 104, environmental sensors 106, and output devices 108 for different locations. For example, at the home of user 110, sleep sensors 104 may include sensors for heart rhythm, respiration, and movement. In this example, at the home of user 110, output devices 108 may include earbuds and a temperature-controlled blanket. However, at a workplace dormitory used by user 110 (e.g., if user 110 is a firefighter and sleeps some of the time at a fire station, or if user 110 is a truck driver and sleeps some of the time in a sleeper cab of a truck), sleep sensors 104 may include sensors for heart rhythm, movement, and ocular movement. In this example, at the workplace dormitory, output devices 108 may include only earbuds. Thus, sleep assistance unit 212 may use a first location-specific model to determine a sleep state of user 110 when user 110 is at a first location (e.g., home) and may use a second location-specific model to determine a sleep state of user 110 when user 110 is at a second location (e.g., a workplace dormitory). In this example, the first location-specific model may be adapted to determine the sleep state based on sleep data from the sleep sensors available at the first location and the second location-specific model may be adapted to determine the sleep state based on sleep data from the sleep sensors available at the second location.

In some examples, there may be different output devices at different locations. Accordingly, action selection unit 404 may use a first location-specific model associated with a first location to identify actions that can be performed by output devices at the first location. Action selection unit 404 may use a second location-specific model associated with a second location to identify actions that can be performed by output devices at the second location.

In some examples, there may be a coordinating device (e.g., a mobile hub) that is configured to obtain data from devices at a location of user 110. The coordinating device may establish communication links (e.g., wireless or wire-based communication links) with devices at a particular location that include one or more of sleep sensors 104, environmental sensors 106, and output devices 108. In other words, the coordinating device may be configured to connect to a surrounding sensor network. In some examples, to obtain the data from the devices at the location and provide instructions to output devices, the coordinating device may access applications (e.g., via APIs) operating on devices that host sleep sensors 104 and environmental sensors 106 and to output devices 108.

The coordinating device may perform the functions of computing system 102 (FIG. 1) or may provide sleep data 216 and environmental data 218 to computing system 102. Moreover, the coordinating device may perform actions determined by action selection unit 404 or may relay instructions to perform actions to output devices at the location. User 110 may bring the coordinating device with user 110 as user 110 moves from location to location. The coordinating device may be an earbud, mobile device, wearable device, or other type of device.

In some examples, machine-learned models used by disturbance prediction unit 400, event classification unit 402, and/or action selection unit 404 may generate predictions based on user profile information. In other words, inputs to the machine-learned models may include the user profile information. The user profile information may include information about user 110 in addition to sleep data 216 and/or environmental data 218. For example, the user profile information may include one or more of a demographic profile of user 110 (e.g., age, job type, gender, etc.), physical activity information regarding user 110, a medical information regarding user 110, and so on.

In some examples, disturbance prediction unit 400, event classification unit 402, and action selection unit 404 may maintain libraries of stored machine-learned models associated with different types of user profiles, different types of available sensors and output devices, and/or locations. When user 110 begins using system 100 or begins using system 100 at a new location, disturbance prediction unit 400, event classification unit 402, and/or action selection unit 404 may select initial versions of the machine-learned models from the library that are associated with user profiles most similar to the user profile of user 110 and are associated with the types of sensors available to user 110 and/or that are associated with a same or similar location as user 110.

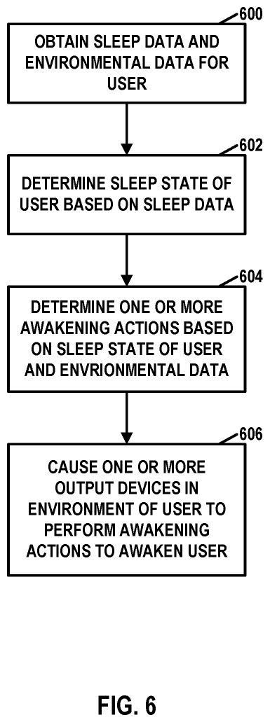

FIG. 6 is a flowchart illustrating an example operation of the processing system in accordance with one or more aspects of this disclosure. The operation shown in this flowchart is provided as an example. In other examples, operations may include more, fewer, or different actions, and/or actions may be performed in different orders. FIG. 6 is explained with reference to FIG. 1 through FIG. 5. However, in other examples, the actions described in FIG. 6 may be performed in other contexts and by other components.

In the example of FIG. 6, computing system 102 may obtain sleep data 216 and environmental data 218 for user 110 (600). Computing system 102 may obtain sleep data 216 and environmental data 218 from sleep sensors 104 and environmental sensors 106, e.g., as described elsewhere in this disclosure. Sleep data 216 may include respiration data that describes respiration of user 110, movement data that describes movement of user 110, cardiac data that describes cardiovascular activity of user 110, body temperature data that describes a body temperature of user 110, blood pressure data that describes a blood pressure of user 110, ocular movement data that describes ocular movement of user 110, and/or other information from which the sleep state of user 110 may be determined.

Additionally, computing system 102 may determine a sleep state 224 of user 110 based on sleep data 216 (602). For instance, as described elsewhere in this disclosure, sleep analysis unit 210 may use ML model 213 (e.g., a neural network) that predicts sleep state 224 based on sleep data 216 (e.g., based on processed sleep data 220).

Computing system 102 may determine one or more awakening actions based on sleep state 224 of user 110 and environmental data 218 (604). For example, event classification unit 402 may determine that an alarm event is occurring. The alarm event may be an event that requires user 110 to awaken. In this example, based on the occurrence of the alarm event, action selection unit 404 may determine one or more awakening actions or actions to assist the sleep of user 110 based on the sleep state 224 of user 110 and environmental data 218. For instance, action selection unit 404 may adjust an output volume, temperature, illumination level, or other output parameter based on the sleep state 224 and the environmental data 218, e.g., as described elsewhere in this disclosure.

For instance, action selection unit 404 may use an equation, such as Equation 1, to calculate an output level (e.g., output volume, temperature, illumination level, or other parameter) based on sleep state 224 and environmental data 218. The equation includes a weight (e.g., A value) for the sleep state and weights (e.g., λ values) for the environmental data. In some examples, the weight for the sleep state and the weights for the environmental data are specific to a location of user 110.

In some examples, computing system 102 obtains location data that indicates a location of user 110. Computing system 102 may determine the one or more awakening actions based on sleep state 224 of user 110, environmental data 218, and the location of user 110. For instance, computing system 102 may obtain data indicating alarm condition preferences established for user 110. For instance, computing system 102 may receive indications of user input specifying alarm-condition preference for user 110. For each of one or more locations, the alarm-condition preferences established for user 110 for the location may specify sets of alarm conditions for the location. Based on computing system 102 obtaining data indicating that user 110 is at a particular location, event classification unit 402 may use the alarm condition preferences established for user 110 for the particular location to determine whether an alarm event is occurring. Thus, in some examples, computing system 102 may determine, based on data for the particular location, whether the alarm event is occurring.

In some examples, user agnostic alarm conditions may be established for one or more locations. User agnostic alarm conditions for a location may be specific to the location but not specific to individual users. For example, computing system 102 may evaluate user agnostic alarm conditions to determine, for any users sleeping at the particular location, whether an alarm event is occurring. An example user agnostic alarm condition may be established with respect to sleeping berths on a vehicle (e.g., train, ship, airplane, etc.). In this example, the user agnostic alarm condition may specify that an alarm event is occurring when the vehicle is within a specific distance to the destination (or an estimated time of arrival of the vehicle at the destination is less than a specified amount). In another example, a user agnostic alarm condition for a nurse sleeping station at a care facility may specify that an alarm event is occurring when a patient at the care facility is experiencing a health event, or when a patient is incoming to the care facility.

Furthermore, computing system 102 may cause one or more output devices 108 in an environment of user 110 to perform the one or more awakening actions to awaken user 110 (606). For example, device control unit 214 may send instructions to one or more of output devices 108 to adjust an output volume, noise cancelation level, temperature, illumination level, and so on. For instance, to awaken user 110, device control unit 214 may decrease the output volume of sleep-assistance sounds, increase output volume of awakening noises, reduce noise cancelation, increase temperature, increase illumination, and so on.

In this disclosure, ordinal terms such as “first,” “second,” “third,” and so on, are not necessarily indicators of positions within an order, but rather may be used to distinguish different instances of the same thing. Examples provided in this disclosure may be used together, separately, or in various combinations. Furthermore, with respect to examples that involve personal data regarding a user, it may be required that such personal data only be used with the permission of the user.

The following paragraphs provide a non-limiting list of examples in accordance with techniques of this disclosure.

Example 1: A method for managing sleep of a user includes obtaining, by a computing system, sleep data and environmental data for the user; determining, by the computing system, a sleep state of the user based on the sleep data; determining, by the computing system, one or more awakening actions based on the sleep state of the user and the environmental data; and causing, by the computing system, an output device in an environment of the user to perform the one or more awakening actions to awaken the user.

Example 2: The method of example 1, wherein the method further comprises obtaining location data that indicates a location of the user, and wherein determining the one or more awakening actions comprises determining the one or more awakening actions based on the sleep state of the user, the environmental data, and the location of the user.

Example 3: The method of any of examples 1 and 2, wherein determining the one or more awakening actions comprises determining an output level of the output device based on the sleep state of the user and the environmental data.

Example 4: The method of example 3, wherein the output level is one of an output volume, an illumination level, or a temperature.

Example 5: The method of any of examples 3 and 4, wherein determining the output level of the output device comprises applying, by the computing system, an equation to calculate the output level, wherein the equation includes one or more weights for the sleep state and one or more weights for the environmental data.

Example 6: The method of example 5, wherein the one or more weight for the sleep state and the one or more weights for the environmental data are specific to a location of the user.

Example 7: The method of any of examples 5 and 6, wherein the method further comprises applying, by the computing system, a machine learning process to learn the one or more weights for the sleep state and the one or more weights for the environmental data.

Example 8: The method of example 7, wherein applying the machine learning process comprises learning, by the computing system, the weights for the sleep state and the weights for the environmental data using data regarding people other than the user who sleep at a same location as the user.

Example 9: The method of any of examples 1 through 8, wherein the method further comprises determining, by the computing system, whether an alarm event is occurring, and wherein determining the one or more awakening actions comprises, based on a determination that the alarm event is occurring, determining the one or more awakening actions based on the sleep state of the user and the environmental data.

Example 10: The method of example 9, wherein determining whether the alarm event is occurring comprises determining, by the computing system, based on a profile of a location of the user whether the alarm event is occurring.

Example 11: The method of any of examples 1 through 10, wherein the sleep data comprises one or more of: respiration data that describes respiration of the user, movement data that describes movement of the user, cardiac data that describes cardiovascular activity of the user, body temperature data that describes a body temperature of the user, blood pressure data that describes a blood pressure of the user, or ocular movement data that describes ocular movement of the user.

Example 12: A computing system includes one or more storage devices configured to store sleep data and environmental data for a user; and processing circuitry configured to: determine a sleep state of the user based on the sleep data; determine one or more awakening actions based on the sleep state of the user and the environmental data; and cause an output device in an environment of the user to perform the one or more awakening actions to awaken the user.

Example 13: The computing system of example 12, wherein the processing circuitry is further configured to obtain location data that indicates a location of the user, and wherein the processing circuitry is configured to, as part of determining the one or more awakening actions, determine the one or more awakening actions based on the sleep state of the user, the environmental data, and the location of the user.

Example 14: The computing system of any of examples 12 and 13, wherein the processing circuitry is configured to, as part of determining the one or more awakening actions, determine an output level of the output device based on the sleep state of the user and the environmental data.

Example 15: The computing system of example 14, wherein the output level is one of an output volume, an illumination level, or a temperature.

Example 16: The computing system of any of examples 14 and 15, wherein the processing circuitry is configured to, as part of determining the output level of the output device, apply an equation to calculate the output level, wherein the equation includes one or more weights for the sleep state and one or more weights for the environmental data.

Example 17: The computing system of example 16, wherein the processing circuitry is further configured to apply a machine learning process to learn the one or more weights for the sleep state and the one or more weights for the environmental data, wherein the processing circuitry is configured to, as part of applying the machine learning process to learn the one or more weights for the sleep state and the one or more weights for the environmental data, learn the one or more weights for the sleep state and the one or more weights for the environmental data using data regarding people other than the user who sleep at a same location as the user.

Example 18: The computing system of any of examples 12 through 17, wherein the processing circuitry is further configured to determine whether an alarm event is occurring, and wherein the processing circuitry is configured to, as part of determining the one or more awakening actions, determine, based on a determination that the alarm event is occurring, the one or more awakening actions based on the sleep state of the user and the environmental data.

Example 19: The computing system of example 18, wherein the processing circuitry is configured to, as part of determining whether the alarm event is occurring, determine, based on a profile of a location of the user whether the alarm event is occurring.

Example 20: A non-transitory computer-readable storage medium having instructions stored thereon that, when executed, cause processing circuitry to: obtain sleep data and environmental data for the user; determine a sleep state of the user based on the sleep data; determine one or more awakening actions based on the sleep state of the user and the environmental data; and cause an output device in an environment of the user to perform the one or more awakening actions to awaken the user.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processing circuits to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, cache memory, or any other medium that can be used to store desired program code in the form of instructions or data structures and that can be accessed by a computer. Also, any connection is properly termed a computer-readable medium. For example, if instructions are transmitted from a web site, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Functionality described in this disclosure may be performed by fixed function and/or programmable processing circuitry. For instance, instructions may be executed by fixed function and/or programmable processing circuitry. Such processing circuitry may include one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor,” as used herein may refer to any of the foregoing structure or any other structure suitable for implementation of the techniques described herein. In addition, in some aspects, the functionality described herein may be provided within dedicated hardware and/or software modules. Also, the techniques could be fully implemented in one or more circuits or logic elements. Processing circuits may be coupled to other components in various ways. For example, a processing circuit may be coupled to other components via an internal device interconnect, a wired or wireless network connection, or another communication medium.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples have been described. These and other examples are within the scope of the following claims.

What is claimed is:

1. A method for managing sleep of a user, the method comprising:
obtaining, by a computing system, sleep data and environmental data for the user;
determining, by the computing system, a sleep state of the user based on the sleep data;
determining, by the computing system, one or more awakening actions based on the sleep state of the user and the environmental data; and
causing, by the computing system, an output device in an environment of the user to perform the one or more awakening actions to awaken the user.

2. The method of claim 1,
wherein the method further comprises obtaining location data that indicates a location of the user, and
wherein determining the one or more awakening actions comprises determining the one or more awakening actions based on the sleep state of the user, the environmental data, and the location of the user.

3. The method of claim 1, wherein determining the one or more awakening actions comprises determining an output level of the output device based on the sleep state of the user and the environmental data.

4. The method of claim 3, wherein the output level is one of an output volume, an illumination level, or a temperature.

5. The method of claim 3, wherein determining the output level of the output device comprises applying, by the computing system, an equation to calculate the output level, wherein the equation includes one or more weights for the sleep state and one or more weights for the environmental data.

6. The method of claim 5, wherein the one or more weight for the sleep state and the one or more weights for the environmental data are specific to a location of the user.

7. The method of claim 5, wherein the method further comprises applying, by the computing system, a machine learning process to learn the one or more weights for the sleep state and the one or more weights for the environmental data.

8. The method of claim 7, wherein applying the machine learning process comprises learning, by the computing system, the weights for the sleep state and the weights for the environmental data using data regarding people other than the user who sleep at a same location as the user.

9. The method of claim 1,
wherein the method further comprises determining, by the computing system, whether an alarm event is occurring, and
wherein determining the one or more awakening actions comprises, based on a determination that the alarm event is occurring, determining the one or more awakening actions based on the sleep state of the user and the environmental data.

10. The method of claim 9, wherein determining whether the alarm event is occurring comprises determining, by the computing system, based on a profile of a location of the user whether the alarm event is occurring.

11. The method of claim 1, wherein the sleep data comprises one or more of:
respiration data that describes respiration of the user,
movement data that describes movement of the user,
cardiac data that describes cardiovascular activity of the user,
body temperature data that describes a body temperature of the user,
blood pressure data that describes a blood pressure of the user, or
ocular movement data that describes ocular movement of the user.

12. A computing system comprising:
one or more storage devices configured to store sleep data and environmental data for a user; and
processing circuitry configured to:
determine a sleep state of the user based on the sleep data;
determine one or more awakening actions based on the sleep state of the user and the environmental data; and
cause an output device in an environment of the user to perform the one or more awakening actions to awaken the user.

13. The computing system of claim 12,
wherein the processing circuitry is further configured to obtain location data that indicates a location of the user, and
wherein the processing circuitry is configured to, as part of determining the one or more awakening actions, determine the one or more awakening actions based on the sleep state of the user, the environmental data, and the location of the user.

14. The computing system of claim 12, wherein the processing circuitry is configured to, as part of determining the one or more awakening actions, determine an output level of the output device based on the sleep state of the user and the environmental data.

15. The computing system of claim 14, wherein the output level is one of an output volume, an illumination level, or a temperature.

16. The computing system of claim 14, wherein the processing circuitry is configured to, as part of determining the output level of the output device, apply an equation to calculate the output level, wherein the equation includes one or more weights for the sleep state and one or more weights for the environmental data.

17. The computing system of claim 16,
wherein the processing circuitry is further configured to apply a machine learning process to learn the one or more weights for the sleep state and the one or more weights for the environmental data,
wherein the processing circuitry is configured to, as part of applying the machine learning process to learn the one or more weights for the sleep state and the one or more weights for the environmental data, learn the one or more weights for the sleep state and the one or more weights for the environmental data using data regarding people other than the user who sleep at a same location as the user.

18. The computing system of claim 12,
wherein the processing circuitry is further configured to determine whether an alarm event is occurring, and
wherein the processing circuitry is configured to, as part of determining the one or more awakening actions, determine, based on a determination that the alarm event is occurring, the one or more awakening actions based on the sleep state of the user and the environmental data.

19. The computing system of claim 18, wherein the processing circuitry is configured to, as part of determining whether the alarm event is occurring, determine, based on a profile of a location of the user whether the alarm event is occurring.

20. A non-transitory computer-readable storage medium having instructions stored thereon that, when executed, cause processing circuitry to:
obtain sleep data and environmental data for the user;
determine a sleep state of the user based on the sleep data;
determine one or more awakening actions based on the sleep state of the user and the environmental data; and
cause an output device in an environment of the user to perform the one or more awakening actions to awaken the user.
